(19) United States
(12) Patent Application Publication
(10) Pub. No.: US 2021/0118565 A1
(43) Pub. Date: Apr. 22, 2021

(54) MEDICAL DEVICE AND METHOD FOR RECEIVING DATA IN A MEDICAL DEVICE

(71) Applicant: OLYMPUS WINTER & IBE GMBH, Hamburg (DE)

(72) Inventor: Karsten LORENZ, Teltow (DE)

(73) Assignee: OLYMPUS WINTER & IBE GMBH, Hamburg (DE)

(21) Appl. No.: 17/075,990

(22) Filed: Oct. 21, 2020

(30) Foreign Application Priority Data
Oct. 21, 2019 (DE) 10 2019 128 378.7

(51) Int. Cl. G16H 40/67; H04L 29/06; G06F 13/42

(52) U.S. Cl. CPC G16H 40/67; H04L 69/08; H04L 63/1416; H04L 63/0227; G06F 13/4282

(57) ABSTRACT

A medical device including: a control unit which is configured to control functions of the medical device, wherein the control unit includes: a processor and a network interface for exchanging data with other devices via a network, wherein the network interface is configured to exchange data using a first communication protocol and to translate data received via the network into a second communication protocol, and to forward the translated data to the processor. The processor is configured to compare data received from the network interface with permissible and/or expected data, and to reject or ignore data received from the network interface if these do not match permissible and/or expected data. Furthermore, a method for receiving data in a medical device is presented.

The invention relates to a medical device comprising a control unit which is configured to control functions of the medical device, wherein the control unit comprises the following: a processor and a network interface for exchanging data with other devices via a network, wherein the network interface is configured to exchange data using a first communication protocol and to forward the data received via the network to the processor.

The invention also relates to a method for receiving data in a medical device.

Nowadays, modern medical devices increasingly have electronically controlled functions. These functions include on the one hand the medical device function itself, such as the generation of electrosurgical therapy signals, and on the other hand the collection and communication of operating and/or diagnostic data.

Such medical devices are equipped with an electronic control unit to control the corresponding functions. The core of such a control unit is usually a microprocessor.

In order to be able to exchange operating and diagnostic data with other devices, the control unit usually also comprises a network interface. Modern medical devices can also receive relevant control data for the actual medical function via the network interface, such as parameters for new or adapted diagnostic or treatment methods.

The exchange of data from medical devices via a network usually takes place using standardized communication protocols. This ensures that medical devices of different types and from different manufacturers can be networked with one another without any problems. Common protocols include Ethernet, WLAN and Bluetooth.

In the event that the control unit's microprocessor cannot directly process a protocol used for data exchange, the network interface can translate the first communication protocol into a second communication protocol which the microprocessor can process.

Communication protocols are mostly designed for the most extensive functionality possible. They enable not only the pure transmission of data, but also a direct influence on connected systems up to complete remote control. Medical devices comprising a network interface could therefore be influenced by a potential invader, which must be prevented under any circumstances.

To protect against unauthorized interference via the network, common operating systems which are also used in medical devices provide protective functions in the form of a firewall.

However, due to their complex structure, these protective functions usually have errors that can be used by invaders to bypass the firewall. The operating systems therefore have to be updated regularly in order to correct newly discovered errors. Usually, however, new errors are found and exploited by invaders first, so that medical devices are always exposed to a certain risk of attack despite regular updates.

The object is therefore to provide a medical device which is improved with regard to the problems described.

According to one aspect of the invention, this object is achieved by a medical device comprising a control unit which is configured to control functions of the medical device, wherein the control unit comprises: a processor and a network interface for exchanging data with other devices via a network, wherein the network interface is configured to exchange data using a first communication protocol and to translate data received via the network into a second communication protocol, and to forward the translated data to the processor, which is further developed in that the processor is configured to compare data received from the network interface with permissible and/or expected data, and to reject or ignore data received from the network interface if these do not match permissible and/or expected data.

In particular a communication protocol that is not intended and/or suitable for directly influencing connected systems can be used for the communication between the network interface and the processor. The additional plausibility or admissibility check of the received data creates an additional level of security, so that reliable protection against unauthorized interference with the medical device is achieved. A further safety barrier is set up between the medical device and the network. In addition to limiting the possibility of influencing the functional scope of the second communication protocol, a limitation to precisely those types of communication content is achieved, which were foreseen by the manufacturer of the medical device. Thus, an invader cannot access any weak points that may be present in the second communication protocol as long as the corresponding communication content is not explicitly contained in the permissible data. In a possible embodiment of a medical device according to the invention, the processor can execute a filter algorithm by means of which the received data is compared with permissible and/or expected data. Such a filter algorithm can be implemented with most basic data processing functions of the processor and is therefore particularly immune to hidden hardware and firmware errors in the processor or errors in the operating system.

In a preferred embodiment of a medical device according to the invention, the network interface can comprise a microcontroller which is configured to translate data received via the network into the second communication protocol and to send these to the processor, and/or to translate data received from the processor into the first communication protocol and to send these to the network.

The microcontroller thus acts as a translator between the first communication protocol and the second communication protocol. Communication content received via the network, which, for example, uses remote control functions provided in the first communication protocol, is translated into harmless communication content.

In one possible embodiment of the invention, the medical device can comprise a housing, and the network interface can be arranged in the housing. The network interface can be arranged and designed in the housing in such a way that a connection to the network interface can be established from outside the housing. A corresponding open design of the network interface is made possible by the security architecture of the network interface and the control unit according to the invention, without the security of the control unit being weakened against unauthorized interference as a result.

In an advantageous embodiment of a medical device according to the invention, the second communication protocol can be a pure data exchange protocol. With such a communication protocol, undesired interference with the medical device can be rendered practically impossible.

The second communication protocol can be an SPI protocol. The SPI is particularly well suited for pure data transmission.

In a further embodiment of a medical device according to the invention, the control unit can furthermore comprise a memory element, and a list of expected and/or permissible data can be stored on the memory element. With a corresponding configuration of the medical device, it is possible to adapt the permissible data stored on the memory element to changed and/or newly developed functions of the medical device.

A medical device according to the invention can be an electrosurgical high-frequency generator and/or an ultrasonic generator.

According to a further aspect of the invention, the object is achieved by a method for receiving data in a medical device, comprising the steps of: receiving a data packet from a network using a first communication protocol, converting the data packet into a second communication protocol that differs from the first communication protocol, forwarding the data packet to a processor, checking whether the data packet corresponds to permissible or expected data, and rejecting or ignoring the data packet if it does not correspond to permissible or expected data.

In a possible embodiment of a corresponding method, the data packet can be checked using a filter algorithm executed by the processor.

With regard to further explanations of the method and thus achievable advantages and effects, reference is made explicitly to the above.

The invention is explained in more detail below with reference to exemplary drawings. The exemplary embodiments shown in the drawings are intended to contribute to a better understanding of the invention without being restrictive.

In the drawings:

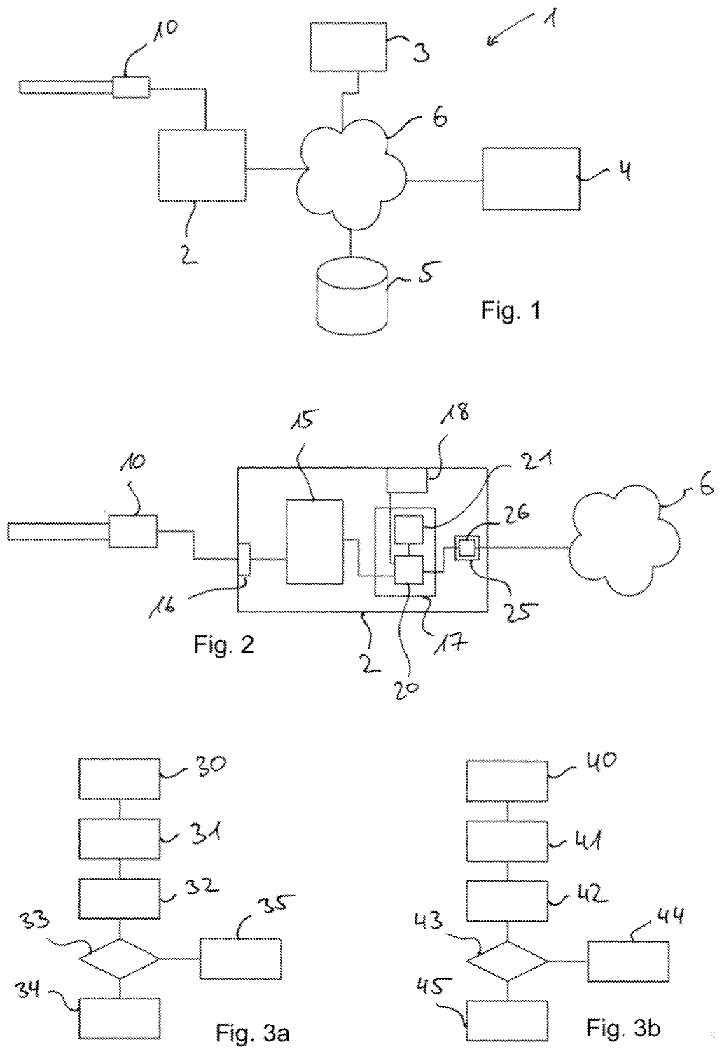

FIG. 1 shows a medical device system,

FIG. 2 shows a medical device,

FIGS. 3a and 3b show a method for receiving data in a medical device.

In FIG. 1, a medical device system 1 is shown with a plurality of medical devices 2, 3, 4, 5, which are connected via a network 6. The network 6 can comprise a local area network (LAN) or a wide area network (WAN), for example the internet.

In the example shown, the medical device 2 is an electrosurgical generator to which a surgical instrument 10 is connected.

The further medical devices 3, 4, 5 can include, for example, an insufflator, a medical device controller and/or a patient database.

The connection of the medical devices 2, 3, 4, 5 to the network 6 is implemented via standardized communication protocols, for example via a wired Ethernet connection or via a wireless WLAN or Bluetooth connection.

The basic structure of the medical device 2 is shown schematically in FIG. 2. The medical device 2 comprises a medical functional module 15 which is configured to carry out a medical function. The functional module 15 can be, for example, a surgical high-frequency generator.

The surgical instrument 10 is connected to the functional module 15. An isolating module 16, for example an isolating transformer, is provided for galvanic isolation of a patient circuit containing the surgical instrument 10 from a supply voltage.

The medical device 2 further comprises a control unit 17 which controls the functional module 15. The control unit is connected to a user interface 18, which can comprise, for example, switches, rotary actuators, foot pedals and/or a touch screen. A user can, for example, set treatment parameters and/or start or end a treatment via the user interface 18.

The control unit 17 comprises a processor 20 and a memory element 21 on which, among other things, program instructions for the processor 20 and control data for the functional module 15 are stored.

The processor 20 can be connected directly to the network 6, for example by means of an integrated Ethernet interface. Here, however, the problem arises that the Ethernet protocol has extensive functions to influence the processor 20, up to and including total remote control. Some of the corresponding functions intervene directly in the operation of the processor 20 and are therefore difficult to control by means of an operating system, which is, after all, just a program running on the processor 20. It is also possible that the hardware and/or the firmware of the processor 20 have weak points which can be exploited by certain functions of the communication protocol to bypass the control functions of an operating system.

In order to protect the processor 20 from unwanted interference via the Ethernet interface, the medical device 2 has a separate Ethernet interface 25, which is connected on the one hand to the processor 20 and on the other hand to the network 6.

The connection between the Ethernet interface 25 and the processor 20 is implemented via a communication protocol which offers significantly fewer possibilities for undesired interference with the processor 20 via the network 6 than the Ethernet communication protocol. The communication between the processor 20 and the Ethernet interface 25 preferably takes place by means of a pure data protocol, such as the SPI protocol or the I²C protocol.

For this purpose, the Ethernet interface 25 comprises a microcontroller 26, which converts data received via the Ethernet connection into the corresponding data protocol and then forwards it to the processor 20. In the opposite direction, data that the microcontroller 26 receives in the data protocol from the processor 20 is translated by the microcontroller 26 into the Ethernet protocol and sent to the network 6.

During the translation from the Ethernet protocol into the data protocol, control commands that directly affect the processor 20 are lost or are translated into harmless data packets that cannot directly affect the processor 20.

In order to create a further level of protection between the processor 20 and the network 6, a program runs on the processor 20 that compares the data packets received from the network 6 with permissible or expected data.

If a received data packet does not correspond to the permissible or expected data, the program can ignore the corresponding data. In this way, unauthorized data with which an attacker could attempt to influence the function of the medical device 2 is filtered out.

The comparison of received data with permissible data can take place via what is known as a positive list, which is stored on the memory element 21. In this case, all permissible types of data packets are listed in the positive list, and the processor 20 only processes data which correspond to the data types in the positive list. The data type can be defined by a header of the data, i.e. a fixed data structure at the beginning of each data packet.

The comparison of received data with permissible data can alternatively or additionally take place via a negative list which is stored on the memory element 21. In this case, illegal types of data packets are stored in the list, and the processor 20 processes all data which do not correspond to a data type shown in the list.

Methods for receiving data in the medical device 2 are shown in FIGS. 3a and 3b.

FIG. 3a shows a method in which, in step 30, a data packet is received from the network. In step 31, the data packet is translated from the Ethernet protocol into the SPI protocol. In step 32, the translated data packet is sent to the processor 20.

In step 33, it is checked whether the received data packet corresponds to expected or permissible data. For this purpose, the data packet is compared with a positive list which contains all permissible data types. If the data packet corresponds to a data type recorded in the positive list, the data packet is processed by the processor 20 in step 34. Otherwise, the data patron is rejected or ignored in step 35.

FIG. 3b shows a further method for receiving data. Here, a data packet is received in step 40, translated from the Ethernet protocol into the SPI protocol in step 41, and sent to the processor 20 in step 42.

In step 43, the data packet is compared with a negative list in which non-permissible data types are stored. If the data packet corresponds to a data type stored in the negative list, the data packet is rejected in step 44. Otherwise, it is processed in step 45.

When processing data packets in steps 32 or 44, the content of the data packets can be converted into control commands which intervene in the functioning of the processor 20 by a program executed on the processor 20. However, this is always carried out under the full control of the filter algorithm running on the processor. Using the positive and/or negative lists already described and, if necessary, other criteria, it can be decided whether a corresponding action is permissible. The filter algorithm can block the execution of an action if a transferred amount of data does not match an action to be carried out with the data, for example if the transferred amount of data is larger than a memory area in which the data is to be stored. The filter algorithm can also take into account an operating state of the medical device and only allow certain actions in a certain operating state.

1. A medical device comprising:
a control unit which is configured to control functions of the medical device, wherein the control unit comprises the following:
processor, and
a network interface for exchanging data with other devices via a network wherein
the network interface is configured to exchange data using a first communication protocol, and
to translate data received via the network into a second communication protocol and to forward the translated data to the processor, wherein
the processor is configured to compare data received from the network interface with permissible and/or expected data, and to reject or ignore data received from the network interface if these do not match permissible and/or expected data.

2. The medical device according to claim 1, wherein the processor executes a filter algorithm by means of which the received data is compared with permissible and/or expected data.

3. The medical device according to claim 1, wherein the network interface comprises a microcontroller which is configured
to translate data received via the network into the second communication protocol and to send it to the processor, and/or
to translate data received from the processor into the first communication protocol and to send it to the network.

4. The medical device according to claim 1, wherein the medical device comprises a housing and that the network interface is arranged in the housing.

5. The medical device according to claim 4, wherein the network interface is arranged and designed in the housing in such a way that a connection to the network interface can be established from outside the housing.

6. The device according to claim 1, wherein the second communication protocol is a pure data exchange protocol.

7. The medical device according to claim 1, wherein the second communication protocol is an SPI protocol.

8. The medical device according to claim 3, wherein the control unit further comprises a memory element and that a list of expected and/or permissible data is stored on the memory element.

9. The medical device according to claim 1, wherein the medical device is an electrosurgical high-frequency generator and/or an ultrasonic generator.

10. A method for receiving data in a medical device, comprising the steps of:
receiving a data packet from a network using a first communication protocol,
converting the data packet into a second communication protocol that differs from the first communication protocol,
forwarding the data packet to a processor,
checking whether the data packet corresponds to permissible or expected data, and
rejecting or ignoring the data packet if it does not correspond to permissible or expected data.

11. The method according to claim 10, wherein the data packet is checked using a filter algorithm executed by the processor.
